(19) United States
(12) Patent Application Publication
(10) Pub. No.: US 2006/0282035 A1
(43) Pub. Date: Dec. 14, 2006

(54) Cyanoacrylate adhesive applicator swab

(76) Inventors: Peter Battisti, Alpharetta, GA (US); Joe Rach, Suwanee, GA (US); James Quinn, Portola Valley, CA (US)

Correspondence Address: LEACH PATENT SERVICES, 5 EAST ALDINE DRIVE, HOCKESSIN, DE, 19707, US

(21) Appl. No.: 11/149,794

(22) Filed: Jun. 10, 2005

(51) Int. Cl. A61M 35/00

(52) U.S. Cl. 604/1; 424/78.27

(57) ABSTRACT

A swab applicator for containing and dispensing cyanoacrylate adhesives is disclosed. The device comprises a tube applicator with a valve to contain the cyanoacrylate until use. The valve can be a ball, a bead or a capsule, or it can be an oblong object having a channel closed at one end. The device further comprises an absorbent material which is saturated by cyanoacrylate for application upon opening of the valve. The device can be heat sterilized using dry heat sterilization without rupturing prematurely from pressure generated during the sterilization process.

### BACKGROUND OF THE INVENTION

1. Field of the Invention

The present invention relates to a disposable device for dispensing cyanoacrylate compositions, a method for dispensing said compositions and methods for manufacturing said devices.

2. Background

It is known to use 2-cyanoacrylate esters as adhesives for bonding tissue in medical or surgical procedures performed upon the human or animal body. 2-cyanoacrylate esters polymerize rapidly, and often instantaneously, upon contact with tissue or body fluid. In these applications, the adhesive composition can be used to close wounds, as well as for covering and protecting surface injuries such as lacerations, abrasions, burns, sores and other open surface wounds. These are commonly applied from small tubes of the monomeric adhesive. As the small tube's contents are typically not consumed, the container must either have a re-closable cap, or else the remaining contents must be disposed of. However, the high reactivity of the cyanoacrylates requires that the tube be re-closed quickly to avoid polymerizing the entire tube. The 2-cyanoacrylate is often dispensed onto a cotton swab and then applied to the wound. As the surface of the cotton swab often has enough moisture to initiate polymerization of the cyanoacrylate monomer, it can be difficult to apply the cyanoacrylate to the swab, close the container, and pick up the swab and apply the adhesive with enough time before the adhesive starts to polymerize.

U.S. Pat. No. 6,779,938 discloses a disposable device wherein the composition to be dispensed is contained in a sealed container within the device, which has a means to open the container, and an applicator end.

U.S. Pat. No. 5,100,028 discloses a flexible fluid dispenser which is sealed at one end and sealed at the second end with a shaped seal which is said to focus pressure forces generated within the vessel when external pressure is applied, to open the seal in the region of the seal in which the forces are focused in a predictable fashion.

Puritan Medical Products markets a swab device under the name Liquishield popule, which comprises a tubular container containing a cyanoacrylate adhesive wherein gentle squeezing pressure on the tubular container causes hydrostatic pressure to build up throughout the tube and causes a rupture at the weakest point. This weak point has been deliberately created by melting a thin spot in the wall of the tube. A foam applicator pad covers the weak spot and as the adhesive escapes it can be spread using the applicator pad. The mechanism to generate the weakness appears to be intrinsically variable, since the pressure needed to release the fluid is very variable, with some Popules™ requiring significant force to open, and others bursting in transit.

### BRIEF SUMMARY OF THE INVENTION

The present invention comprises a disposable device for dispensing cyanoacrylate compositions. In one embodiment of the invention, the device comprises a swab applicator which contains a cyanoacrylate composition. The swab applicator device of the invention is closed at one end and covered with a swab applicator with the cyanoacrylate composition contained by a valve which can be readily opened when desired. The device may be heat sterilized. The features and advantages of the device and compositions of the present invention will become apparent from the following more detailed description.

### BRIEF DESCRIPTION OF THE DRAWINGS

For a more complete understanding of the features and objects of the invention, reference should be made to the following detailed description of the invention and the accompanying drawings, in which:

FIG. 1 is a lateral view of one embodiment of the invention prior to the swab being sealed.

FIG. 2 is a lateral cutaway view of one embodiment of the invention prior to the swab being sealed.

FIG. 3 is a top lateral view of one embodiment of the invention after sealing.

FIG. 4 is a side lateral cutaway view of one embodiment of the invention after sealing.

FIG. 5 is a top view one embodiment of the invention illustrating where the valve can be snapped to open the valve.

FIG. 6 is a lateral cutaway view of the invention after the valve has been opened.

FIG. 7 is an expanded view of the circled area of FIG. 6.

FIG. 8 is a lateral view of another embodiment of the invention prior to the swab being sealed.

FIG. 9 is a lateral cutaway view of another embodiment of the invention prior to the swab being sealed.

FIG. 10 is a top lateral view of another embodiment of the invention after sealing.

FIG. 11 is a side lateral cutaway view of another embodiment of the invention after sealing.

FIG. 12 is a top view of another embodiment of the invention illustrating where the valve can be crushed to open the valve.

FIG. 13 is a lateral cutaway view of another embodiment of the invention after the valve has been opened.

### DETAILED DESCRIPTION OF THE INVENTION

FIGS. 1 and 2 are lateral and cutaway views of one embodiment of an applicator 100 in accordance with the invention. Applicator 100 comprises a hollow tube 101 with one end, 103, being open after filing with a cyanoacrylate composition, with a second end being covered by an absorbent material, 102. The cyanoacrylate composition, 110, is contained within the hollow tube housing by a valve, 104. The valve can be a cylinder which comprises a channel which runs longitudinally down the cylinder. The channel is sealed at one end.

When the valve is inserted into the hollow tube, it is oriented so that the sealed end of the channel is towards the second end which is covered with the absorbent material. In order to provide a liquid tight seal, the valve is sealed to the interior wall of the hollow tube, 105, prior to filling, at the end of the valve nearest the first end where the valve is inserted, and the device filled with cyanoacrylate composition. The method of sealing the valve to the interior wall can be either heat sealing, or via ultrasonic sealing. To form this seal, a sealing mechanism presses the wall of the hollow tube together with the valve to contact the two surfaces together. The sealing mechanism then applies to the hollow tube sufficient heat or ultrasonic energy to cause the vessel walls to melt. After the heat or ultrasonic energy is no longer applied, the walls of the tube fuse together with the valve to form the seal.

FIGS. 3 and 4 are lateral and cutaway views of the applicator after the tube is sealed at the first end, 103.

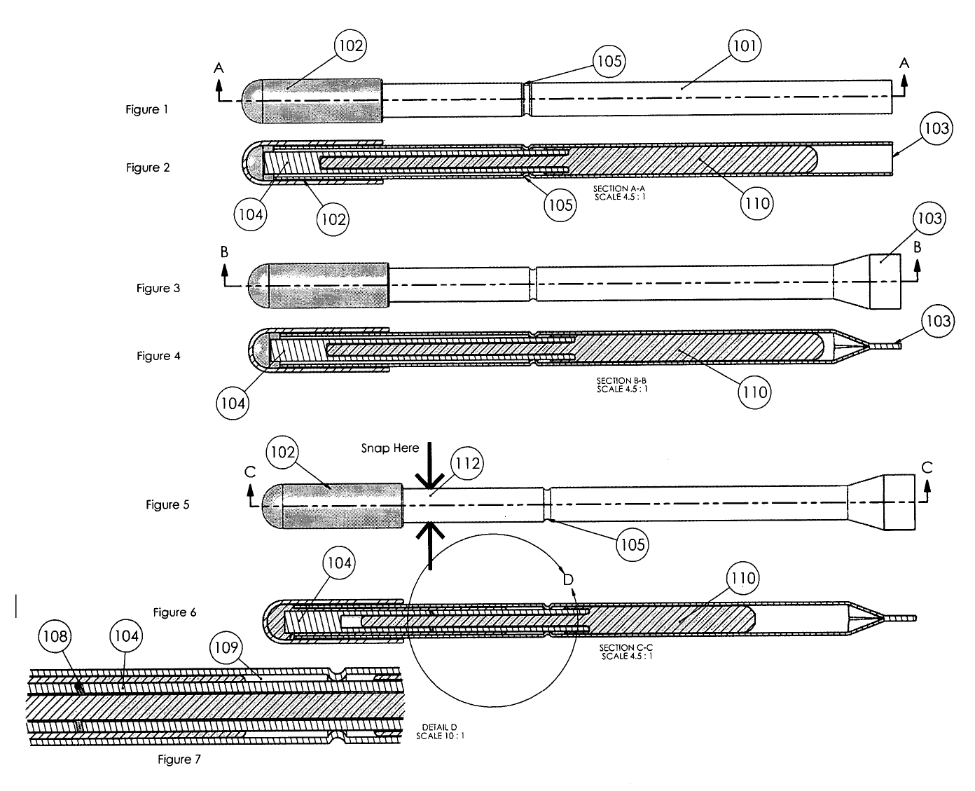

To open the valve and release the contents of the swab, the valve, 104, of the applicator is snapped or bent below the heat seal to open it at 112, as illustrated in FIG. 5. The valve breaks and ruptures into two pieces. This allows the cyanoacrylate composition to flow through the longitudinal channel in the valve, through the leak path 108, into the open space 109 between the swab body 101 and the valve 104, and into the absorbent material covering the end of the applicator as illustrated in FIG. 6, and expanded view FIG. 7.

The embodiment depicted in FIGS. 1-7 of the present invention utilizes a cotton swab applicator with a valve manufactured and sold by Swabplus Inc. in Pomona, Calif.

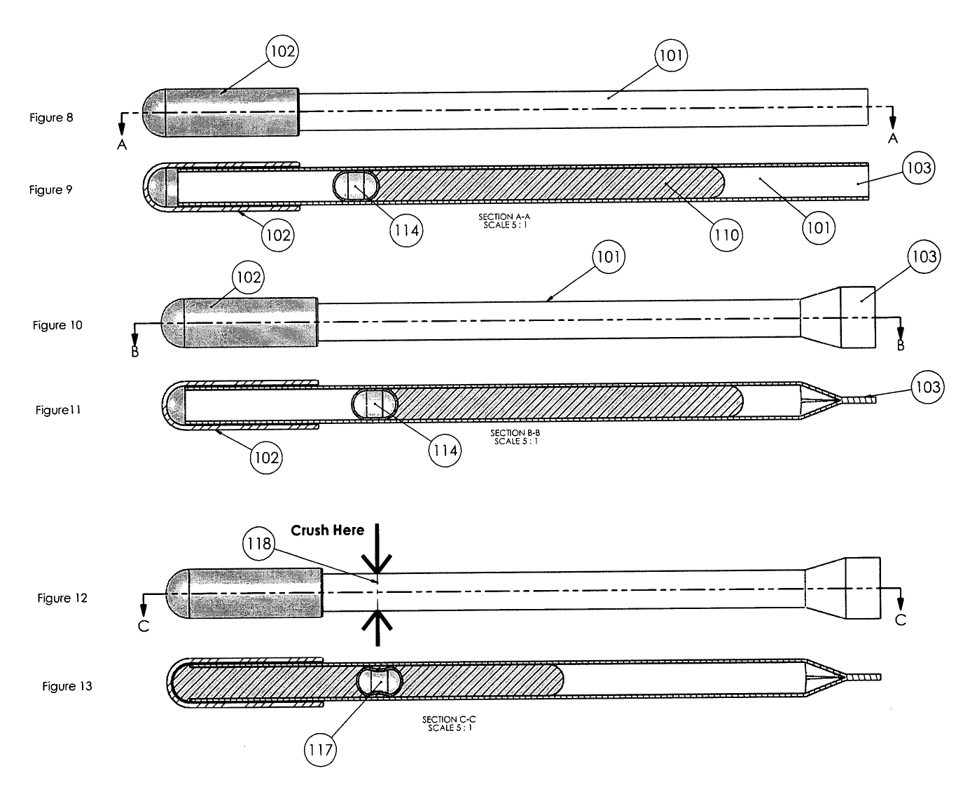

FIGS. 8 and 9 are lateral and cutaway views of another embodiment of an applicator 100 in accordance with the invention. Applicator 100 comprises a hollow tube 101 with one end, 103, being open after filing with a cyanoacrylate composition, with a second end being covered by an absorbent material, 102. The cyanoacrylate composition, 110, is contained within the hollow tube housing by a valve, 114, which is a ball, a bead or a capsule. In order to provide a liquid tight seal, the valve is sealed to the inner wall of the hollow tube by heat or ultrasonic means prior to filling. Alternatively, the valve may designed to be pressure fit into the hollow tube.

FIGS. 10 and 11 are lateral and cutaway views of the applicator after the tube is sealed at the first end, 103.

FIGS. 12 and 13 illustrate how the valve, 114, of the applicator is compressed or crushed to open the valve and release the contents of the applicator. The valve 114 is hollow, and is constructed of a non-rubber material. Thus when the valve is compressed or deformed at 118 in FIG. 12, it does not recover, but rather permanently deforms or ruptures, 117. This allows the cyanoacrylate composition to flow through the tube and into the absorbent material as illustrated in FIG. 13.

It is important that the cyanoacrylate composition contained within the hollow tube of the applicator be protected from moisture, so that polymerization of the cyanoacrylate is not initiated prior to use. Suitable materials of construction for the hollow tube of the applicator should be capable of being a barrier to the diffusion of moisture into the device prior to use. Suitable materials for the hollow tube 101 and valves 104 and 114 include polyolefins such as high density polyethylene (HDPE), polyethylene, polypropylene and the like. High density polyethylene (HDPE) and polypropylene are preferred. High density polyethylene (HDPE) is most preferred. The term high density polyethylene (HDPE) as used herein refers to high density homopolymers of ethylene, and high density copolymers of ethylene with alpha olefins. The term polypropylene as used herein refers to homopolymers of propylene, as well as copolymers of propylene with ethylene and other alpha olefins. The polyolefin may also be fluorinated to prevent the entry of moisture and improve product stability.

The cyanoacrylate composition 110, comprises a cyanoacrylate monomer or monomers which can be selected from the group consisting of alkyl 2-cyanoacrylate, alkenyl 2-cyanoacrylate, alkoxyalkyl 2-cyanoacrylate, or carbalkoxyalkyl 2-cyanoacrylate. The alkyl group of the cyanoacrylate monomer or monomers preferably has 1 to 16 carbon atoms, and includes cycloalkyl functionality. Suitable cyanoacrylates include for example methyl 2-cyanoacrylate, ethyl 2-cyanoacrylate, n-propyl 2-cyanoacrylate, iso-propyl 2-cyanoacrylate, n-butyl 2-cyanoacrylate, iso-butyl 2-cyanoacrylate, hexyl 2-cyanoacrylate, n-octyl 2-cyanoacrylate, 2-octyl 2-cyanoacrylate, 2-methoxyethyl 2-cyanoacrylate, 2-ethoxyethyl 2-cyanoacrylate and 2-propoxyethyl 2-cyanoacrylate. Preferred cyanoacrylates include n-butyl 2-cyanoacrylate, n-octyl 2-cyanoacrylate and mixtures thereof.

The cyanoacrylate compositions of the present invention can be stabilized against premature polymerization with anionic and free-radical polymerization inhibitors. Anionic polymerization inhibitors, known in the art include soluble acidic gases (for example sulfur dioxide), and phosphoric, carboxylic and organic sulphonic acids. Free-radical polymerization inhibitors include hydroquinone, t-butyl catechol, hydroxyanisole, butylated hydroxyanisole and butylated hydroxytoluene. Preferred polymerization inhibitors include butylated hydroxyanisole, hydroxyanisole, butylated hydroxytoluene, and mixtures thereof. Free-radical polymerization inhibitors can be used at levels from about 10 ppm to about 200 ppm, depending on the particular polymerization inhibitor. Typical amounts of free-radical polymerization inhibitors can be from about 20 to about 100 ppm.

The applicators of the device can be filled using commonly available filling equipment. Cyanoacrylate monomers are very sensitive to moisture and other contaminants. Accordingly, filling equipment must be kept scrupulously clean and free of contaminants. Any pressurized air used to drive pumps or pressurize liquid reservoirs must be scrupulously dried and filtered to remove contamination. In addition to air pressure driven filing equipment, positive displacement pumps may also be utilized. Positive displacement pumps are preferred.

After filing the device with a cyanoacrylate composition, the end 103 is closed by one of several closing means. These include heat sealing to press the end of the tube together into a flat surface and then fuse them through application of sufficient heat to fuse the polyolefin, and ultrasonic welding wherein the end of the tube is pressed flat and then fused through application of sufficient ultrasonic energy to melt the walls of the tube. After the heat or ultrasonic energy is removed, the surfaces fuse together.

The applicator device of the invention may be sterilized. Cyanoacrylate compositions can be sterilized either by exposure to gamma irradiation, or by dry-heat sterilization. U.S. Pat. 6,136,326 and U.S. patent application Ser. No. 10/944,635 disclose dry-heat sterilization of cyanoacrylate adhesive compositions and their disclosures are herein incorporated by reference. Applicator devices of the invention are capable of withstanding the pressures generated during the sterilization process without rupturing or leaking.

The following examples are offered to illustrate embodiments of the invention, and should not be viewed as limiting the scope of the invention.

### EXAMPLE 1

Polypropylene swabs from Swabplus Inc., located at 9669 Hermosa Avenue Rancho Cucamonga, Calif. 91730 USA, having an oblong valve were filled by hand through a syringe with 0.20 g of a medical cyanoacrylate (FlexAid) available from Chemence Medical Inc., 185 Bluegrass Valley Parkway, Alpharetta, Ga. The open end of the swab was pressed together and heat-sealed with a pair of pliers and a flame. The closed end was fitted with a polyurethane foam tip.

The swap was striped with a magic marker one inch from the closed foam tip. The liquid contents were dispensed from the applicator by bending at the stripe to one side until it snapped. The foam capped tip, filled with the formulated liquid cyanoacrylate was easily dispensed onto the skin surface. In this way a low viscosity formulation could be applied without spillage or dripping of the adhesive onto unwanted areas.

### EXAMPLE 2

Polypropylene swabs as above were filled with formulated liquid bandage, manufactured by Closure Medical Corporation (Lot 092106), sold by Johnson and Johnson as Band-Aid® liquid bandage using a 1 ml pipette. Swabs were filled within a range of 0.153-0.204 g. The open end was pressed together and heat-sealed using a Uline 8″ Impulse Sealer set at 8 for 15-20 seconds. The swabs were then fitted with an activated foam tip obtained from the Band-Aid liquid bandage kit. The liquid bandage was applied to a minor abrasion by squeezing four drops onto the end of the foam end of the activator and compared to the application on a similar wound with the swabplus device by applying as described in example one. Applying the liquid bandage as directed presents an opportunity to spill the liquid. The swab plus applicator permits a controlled application with little chance of spillage. A fill between 0.08-0.10 g is sufficient to saturate the foam tip and cover a minor wound like a paper cut.

### EXAMPLE 3

Polypropylene swabs from Swabplus Inc., located at 9669 Hermosa Avenue, Rancho Cucamonga, Calif. 91730 USA, were dis-assembled to extract the brittle polypropylene valve. These were inserted into LDPE (Ablex 201) and HDPE (Alblex 503) tubes (0.146×0.168×6.0 inch) obtained from Action Technology located at Route 10 East, P.O. Box 111, Clinton, Ill. 61727. The valves were fixed in the device using a soldering iron to seal the outside wall of the valve to the inside wall of the tube. The polyethylene swabs, prepared as above, were filled with a formulated FlexAid liquid adhesive (RCM-11-034), manufactured by Chemence Medical, Inc. using a 1 ml pipette. Swabs were filled in a range of 0.17-0.19 g. The open ends of the swabs were cut to an approximate 3″ length, and the opening pressed together and heat-sealed with a heated pair of pliers. The closed end was fitted with a polyurethane foam tip. The liquid contents were dispensed from the applicator by bending the tube at the valve end to one side until it snapped. The foam capped tip, filled with the formulated liquid cyanoacrylate was easily applied onto the skin surface. In this manner a low viscosity formulation was applied without spillage or dripping of the adhesive onto unwanted areas.

What is claimed as new and desired to be secured by Letters Patent of the United States is:

1. A disposable device for dispensing cyanoacrylate compositions comprising: a swab applicator having a flexible hollow chamber, having a first end and a second end, wherein the first said end is closed, and further comprising:
i. a valve for containing the contents of the device from flowing out of the said second end prior to use, and
ii. an absorbent material attached over the second said end of the device containing the said valve, and
iii. a cyanoacrylate composition disposed within the said hollow chamber.

2. A device as in claim 1 wherein the cross section of the swab applicator is round.

3. A device as in claim 1 wherein the swab applicator device can be made from a polyolefin.

4. A device as in claim 3 wherein the polyolefin is selected from the group consisting of polypropylene and HDPE.

5. A device as in claim 1 wherein the first said end of said hollow chamber of the swab is closed by a closing means for closing and sealing the device.

6. A device as in claim 1 wherein the valve is selected from the group consisting of a ball, a bead and a capsule, each having a cross sectional shape identical to the hollow chamber.

7. A device as in claim 6 wherein the valve is heat sealed to the wall of the hollow tube.

8. A device as in claim 6 wherein the valve is pressure fit to make a tight seal with the inner wall of the hollow tube.

9. A device as in claim 6 wherein the valve is deformable or rupturable by physical pressure.

10. A device as in claim 1 wherein the valve is an oblong shape having:
i. a cross sectional shape identical to the hollow chamber,
ii. a channel running the length of the said valve, said channel being closed at one end,
iii. which is heat sealed to the inner wall of the hollow chamber
iv. which breaks to open an orifice when flexed or bent under pressure.

11. A device as in claim 1 wherein the cyanoacrylate composition comprises at least one 2-cyanoacrylate ester monomer.

12. A device as in claim 11 wherein the at least one 2-cyanoacrylate ester monomer is an alkyl, cycloalkyl, fluoroalkyl, fluorocycloalkyl or fluoroalkoxy 2-cyanoacrylate monomer, or mixture thereof.

13. A device as in claim 12 wherein the alkyl group of the 2-cyanoacrylate is selected from the group consisting of straight chain or branched chain C₄ to C₈ hydrocarbons.

14. A device as in claim 12 wherein the 2-cyanoacrylate ester monomer is selected from the group consisting of n-butyl cyanoacrylate, n-octyl cyanoacrylate and iso-octyl cyanoacrylate.

15. A device as in claim 1 wherein the device is sterilized

16. A device as in claim 15 wherein the device is sterilized through a dry heat sterilization process.

17. A method of dispensing cyanoacrylate compositions for medical applications comprising:
applying physical pressure to a single-use applicator device, wherein said device comprises:
i. a flexible hollow chamber having a first end and a second end wherein the said first end is closed,
ii. a valve for containing the contents of the device from flowing out of the said second end prior to use,
iii. an absorbent material attached over the second said end of the device containing the said valve, and
iv. a cyanoacrylate composition disposed within said hollow chamber, to rupture or deform the said sealing valve at the said second end of the device and release the cyanoacrylate composition;
i. allowing the cyanoacrylate composition to flow through the said valve into the absorbent material, and
ii. applying through dabbing or brush strokes a cyanoacrylate composition to a wound, bum, or skin area.

18. A method as in claim 17 wherein the cross section of the swab applicator is round.

19. A method as in claim 17 wherein the swab applicator can be made from a polyolefin.

20. A method as in claim 19 wherein the polyolefin is selected from the group consisting of polypropylene and HDPE.

21. A method as in claim 17 wherein the first said end of said hollow chamber is closed by a closing means for closing and sealing the device.

22. A method as in claim 17 wherein the valve is selected from the group consisting of a ball, a bead and a capsule, each having a cross sectional shape identical to the hollow chamber.

23. A method as in claim 22 wherein the valve is heat sealed to the inner wall of the hollow chamber.

24. A method as in claim 22 wherein the valve is pressure fit to make a tight seal with the inner wall of the hollow tube.

25. A method as in claim 22 wherein the valve is deformable or rupturable by pressure.

26. A method as in claim 17 wherein the valve is an oblong shape having:
i. a cross sectional shape identical to the hollow chamber,
ii. a channel running the length of the said valve, said channel being closed at one end,
iii. which is heat sealed to the inner wall of the hollow chamber, and
iv. which breaks to open an orifice when flexed or bent under pressure.

27. A method as in claim 17 wherein the cyanoacrylate composition comprises at least one 2-cyanoacrylate ester monomer.

28. A method as in claim 27 wherein the at least one 2-cyanoacrylate ester monomer is an alkyl, cycloalkyl, fluoroalkyl, fluorocycloalkyl or fluoroalkoxy 2-cyanoacrylate monomer, or mixture thereof.

29. A method as in claim 28 wherein the alkyl group of the 2-cyanoacrylate is selected from the group consisting of straight chain or branched chain C₄ to C₈ hydrocarbons.

30. A method as in claim 28 wherein the 2-cyanoacrylate ester monomer is selected from the group consisting of n-butyl cyanoacrylate, n-octyl cyanoacrylate and iso-octyl cyanoacrylate.

31. A method as in claim 17 wherein the device is sterilized prior to use.

32. A method as in claim 31 wherein the device is sterilized through a dry heat sterilization process.

33. A method of manufacturing a cyanoacrylate dispenser comprising:
i. loading a swab device with a cyanoacrylate composition,
ii. closing the device by a closing means for closing and sealing the device;
wherein said swab device comprises:
a) a flexible hollow chamber having a first end and a second end wherein the first said end can be closed,
b) a valve for containing the contents of the device from flowing out of the said second end prior to use, and
c) an absorbent material over the second said end of the device containing the said valve.

34. A method as claim 33 wherein the cyanoacrylate composition comprises at least one 2-cyanoacrylate ester monomer, or mixture thereof.

35. A method as in claim 34 wherein at the at least one cyanoacrylate ester monomer is an alkyl, cycloalkyl, fluoroalkyl, fluorocycloalkyl or fluoroalkoxy 2-cyanoacrylate monomer, or mixture thereof.

36. A method as in claim 35 wherein the alkyl group of the 2-cyanoacrylate is selected from the group consisting of straight chain or branched chain C₄ to C₈ hydrocarbons.

37. A method as in claim 35 wherein the 2-cyanoacrylate ester monomer is selected from the group consisting of n-butyl cyanoacrylate, n-octyl cyanoacrylate and iso-octyl cyanoacrylate.

38. A method as in claim 33 wherein the cyanoacrylate dispenser is sterilized prior to use.

39. A method as in claim 38 wherein the cyanoacrylate dispenser is sterilized through a dry heat sterilization process.

40. A method as in claim 33 wherein the swab device is made from a polyolefin.

41. A method as in claim 40 wherein the polyolefin is selected from the group consisting of polypropylene and HDPE.

42. A method as in claim 33 wherein the cross section of the hollow chamber of the swab device is round.

43. A method as in claim 33 wherein the said valve of the swab device is selected from the group consisting of a ball, a bead or a capsule, each having a cross sectional shape identical to that of the hollow chamber.

44. A method as in claim 43 wherein the said valve of the swab device is heat sealed to the inner wall of the hollow chamber

45. A method as in claim 43 wherein the said valve of the swab device is pressure fit to make a tight seal with the inner wall of the hollow tube.

46. A method as in claim 43 wherein the valve is deformable or rupturable by pressure.

47. A method as in claim 33 wherein the valve is an oblong shape having:
i. a cross sectional shape identical to the hollow chamber,
ii. a channel running the length of the said valve, said channel being closed at one end,
iii. which is heat sealed to the inner wall of the hollow chamber, and
iv. which breaks to open an orifice when flexed or bent under pressure.

48. A method as in claim 33 wherein the swab device is loaded with the said cyanoacrylate composition using a positive displacement pumping device.

49. A disposable fluid dispenser comprising:
i. a flexible hollow chamber, having a first end and a second end, wherein the first said end can be closed,
ii. a valve for containing the contents of the device from flowing out of the said second end prior to use, and
iii. an absorbent material attached over the second said end of the dispenser container the said valve.

50. A dispenser as in claim 49 wherein the cross section of the dispenser is essentially round.

51. A dispenser as in claim 49 wherein the dispenser can be made from a polyolefin.

52. A dispenser as in claim 51 where the polyolefin is selected from the group consisting of polypropylene and HDPE.

53. A dispenser as in claim 49 wherein the first said end of the hollow chamber is closed by a closing means for closing and sealing the device.

54. A dispenser as in claim 49 wherein the valve is selected from the group consisting of a ball, a bead and a capsule, each having a cross sectional shape identical to the hollow chamber.

55. A dispenser as in claim 54 wherein the valve is heat sealed to the inner wall of the hollow chamber.

56. A dispenser as in claim 54 wherein the valve is pressure fit to make a tight seal with the inner wall of the hollow tube.

57. A dispenser as in claim 54 wherein the valve is deformable or rupturable by physical pressure.
